(19) United States
(12) Patent Application Publication
(10) Pub. No.: US 2011/0152249 A1
(43) Pub. Date: Jun. 23, 2011

(54) EVALUATING CENTRAL NERVOUS SYSTEM

(75) Inventors: David Borsook, Concord, MA (US); Lino Becerra, Cambridge, MA (US); David Alan Boas, Winchester, MA (US); Reuben Mezrich, Baltimore, MD (US)

(73) Assignees: THE GENERAL HOSPITAL CORPORATION, Boston, MA (US); THE BRIGHAM AND WOMEN'S HOSPITAL, INC., Boston, MA (US)

(21) Appl. No.: 11/722,145

(22) PCT Filed: Dec. 23, 2005
(86) PCT No.: PCT/US2005/047139

§ 371 Date: Jul. 6, 2009

Related U.S. Application Data
(60) Provisional application No. 60/639,061, filed on Dec. 23, 2004.

(51) Int. Cl. A61K 31/55; A61P 25/00; A61P 25/22; A61P 25/24; A61P 25/18; A61P 29/00; A61B 5/05

(52) U.S. Cl. 514/217; 600/425

(57) ABSTRACT

Evaluating hemodynamic parameters in the cortex can be used as an objective test for CNS activity, e.g., pain.

### CLAIM OF PRIORITY

This application claims priority under 35 USC §119(e) to U.S. Patent Application Ser. No. 60/639,061, filed on Dec. 23, 2004, the entire contents of which are hereby incorporated by reference.

### BACKGROUND

The invention relates to the field of determination of central nervous system (CNS) activity, for example, activity associated with pain. Pain includes sensory and emotional experiences produced by noxious stimuli or by damage to the nervous system (Price DD Science. 2000 288:1769-72.). Recent epidemiological data show that acute and chronic pain is epidemic in the United States. It is estimated that 50% of acute pain is undertreated and that approximately 20% of the adult population suffer from chronic pain conditions (Max MB Ann Intern Med. 1990 113:885-9; Blyth F M et al. Pain. 2001 89:127-34.). There is a need for non-invasive, objective tests for neurological conditions.

### SUMMARY

This disclosure features, inter alia, methods of determining CNS activity in a subject, e.g., to evaluate pain or analgesia or to diagnose a neurological condition, such as a psychiatric disorder. In some embodiments, the methods employ diffuse optical tomography (DOT) to determine a level of activation of a cortical region, e.g., the somatosensory cortex. The methods can be non-invasive, portable, and inexpensive. The methods can be, in some implementations, particularly useful where a subject is unable, or unwilling, to articulate (e.g., while undergoing an operation, while sedated, anesthetized, or unconscious, or while in infancy), where the subject suffers from a neurological condition, such as a psychiatric disorder. Furthermore, the methods may also be used to identify therapeutic interventions or quantify their effects.

In one aspect, the disclosure features a method for measuring a central nervous system (CNS) activity in a subject including determining a level of activation in a cortical region of the subject by performing DOT, wherein the level of activation is indicative of the CNS activity in the subject. The CNS activity may be indicative of a neurological condition or cognition. In some embodiments, the subject may be undergoing a medical, dental, or surgical procedure. The subject may also be anesthetized, and the CNS activity may be indicative of the level of anesthesia. The level of activation may be compared to reference information, e.g., a level of activation (e.g., rendered as an image) of the cortical region of a control subject obtained using DOT or other CNS evaluation technique, wherein the control subject was subject to a different condition, e.g., the subject was not experiencing the CNS activity at the time of performing the evaluation.

This disclosure further features a method for identifying a therapeutic intervention for a neurological condition including administering a test intervention to a subject; performing DOT on the subject in the presence of a stimulus capable of inducing CNS activity indicative of the neurological condition; and determining a level of activation of a cortical region, wherein the level of activation is indicative of the effectiveness of the test intervention as a therapeutic intervention. In one embodiment, the level of activation is determined relative to a control image of the cortical region of a control subject, obtained by DOT during the presence of the stimulus, wherein the test intervention is not administered to the control subject. The stimulus may be noxious, e.g., an acute painful stimulus, or non-noxious.

In another aspect, the invention features a method for identifying a therapeutic intervention for a neurological condition. The method includes: providing a subject experiencing the neurological condition; performing DOT on the subject following administration of a test intervention; and determining a level of activation of a cortical region, wherein the level of activation is indicative of the effectiveness of the test intervention as a therapeutic intervention. The level of activation may be determined relative to a reference information for a reference subject, e.g., a control image of the cortical region of a control subject, obtained by DOT. For example, the reference subject can be a subject to whom the test intervention is not administered.

This disclosure also features a method of managing a neurological condition in a subject including determining a level of activation in a cortical region of a subject by performing DOT on the subject, wherein the level of activation is indicative of the neurological condition; and administering a therapeutic intervention to the subject to maintain or change the level of activation. The outcome of the management may be, for example, maintaining the neurological condition (e.g., anesthesia) or maintaining, adjusting, or initiating treatment of the neurological condition (e.g., pain). The method may be repeated over time, for example, if the subject is under the continuing care of a medical or dental professional, either over long term (e.g., days or weeks) or during a particular task (e.g., during a medical, dental, or surgical procedure). In one embodiment, the level of activation is determined relative to a control image of the cortical region of a control subject, obtained by DOT, wherein the control subject is not experiencing the neurological condition. The subject is, for example, undergoing a medical, dental, or surgical procedure. In one embodiment, the neurological condition includes anesthesia, and the administering maintains anesthesia of the subject, e.g., to prevent chronic pain.

In another aspect, this disclosure features a method for correlating subcortical activity with cortical activity in a subject having a neurological condition including performing a CNS evaluation technique other than DOT on a subject experiencing a neurological condition to determine a level of activation of a subcortical region; performing DOT on a subject experiencing the neurological condition to determine a level of activation of a cortical region; and comparing the level of activation of the subcortical region with the level of activation of the cortical region, thereby correlating subcortical activity with cortical activity. The CNS evaluation technique can be, e.g., magnetoencephalography (MEG), single proton emission tomography (SPECT), positron emission tomography (PET), or functional magnetic resonance imaging (fMRI). The subject evaluated using the CNS evaluation technique other than DOT and the subject evaluated using DOT may be the same individual, or different individuals, e.g., age, gender, race matched individuals. The subcortical activity is, for example, indicative of pain, anesthesia, or a psychiatric disorder.

Exemplary subjects include mammals, e.g., humans and other primates. Exemplary neurological conditions include a psychiatric disorder (e.g., depression or anxiety), pain (e.g., acute or chronic), or anesthesia (local or general). Local anesthesia may cause a direct or indirect effect on CNS activity. To cause a direct effect, CNS structures are anesthetized. Indirect effects are observed when CNS activity is altered by a reduction or lack of sensory information from an anesthetized site. Chronic pain is, for example, chronic nociceptive pain or neuropathic pain. The cortical region observed in the methods described herein is, for example, a region of a parietal lobe, frontal lobe, temporal lobe, or occipital lobe, such as a somatosensory cortex, motor cortex, or lateral prefrontal cortex.

In various embodiments, DOT produces an image of the cortical region from which a level of activation is determined. The level of activation may be determined relative to a reference level, e.g., a control. Reference levels can be based on an evaluation of the same individual as the subject in the methods described herein, or can be based on an evaluation of a different individual, e.g., an age, gender, or race matched individual. In various embodiments, the image from the control subject is a standard control image. A contrast agent may also be administered to the subject prior to detection of a cortical region by DOT.

Exemplary therapeutic or test interventions include administering a compound, a physical stimulus, an electrical stimulus, a thermal stimulus, electromagnetic radiation, or a surgical, medical, or dental procedure.

In one aspect, the disclosure features a method for providing a treatment and evaluating subject for pain or analgesia. In certain embodiments, evaluating the subject for pain or analgesia includes determining level of cortical activation, e.g., in SI. The subject can be evaluate using a method described herein, e.g., by evaluating a hemodynamic parameter, e.g., using DOT.

The subject can be evaluated (e.g., for pain or analgesia) before, during, or after providing the treatment. In one embodiment, the treatment is provided for, e.g., diabetic peripheral neuropathy, posthereptic neuralgia, cancer-associated pain, spinal cord injury, causalgia and reflex sympathetic dystrophy, multiple sclerosis, phantom pain, poststroke, HIV-associated pain, trigeminal neuralgia (tic douloureux) and low-back pain-associated pain. In one embodiment, the treatment includes non-pharmacological methods, e.g., neural blockade (e.g., continuous infusion catheter, neuroablation); neuroaugmentation (e.g., peripheral nerve stimulation, spinal cord stimulation); physical therapy, transcutaneous electrical nerve stimulation (TENS), exercise, physical stimuli (e.g., massage or acupuncture), electrical stimuli, thermal stimuli, electromagnetic radiation, surgical or medical or dental procedure. For example, treatment includes administering pharmacological agents, e.g. over-the counter analgesics and non-steroidal anti-inflammatory drugs (NTHEs) (e.g., Cox2 inhibitors, ibuprofein, aspirin, acetaminophen), anticonvulsants (e.g., carbamazepine, gabapentin, lamotrigine, divalproex, valproate, phenytoin), tricyclic antidepressants (TCAs), topical anesthetic agents (capsaicin, lidocaine, prolicaine, doxepin), oral local anesthetics (mexiletine, tocainide and flecainide), antiarrhythmics, narcotic analgesics, opioids (e.g., tramadol, levorphanol, oxycodone, morphine, butorphanol, meperidine, codeine, propoxyphene, methadone, hydromorphone, fentanyl, oxymorphone, nalbuphine, levomethadyl, hydrocodone, buprenorphine, alfentanil, sufentanil, pentazocine, dezocine), antidepressants (e.g., amitriptyline, nortriptilyline, desipramine, selective serotonin reuptake inhibitors, e.g., duloxetine), N-methyl-D-aspartate-receptor blockers (e.g., ketamine, dextromethorphan, memantine, amantadine), membrane-stabilizing drugs (tocainamide, lidocaine, mexiletine), alpha2 agonists (e.g., clonidine, tizanidine), GABA agonists (e.g., baclofen), corticosteroids (e.g., methylprednisolone, and dexamethasone), and muscle relaxants (e.g., metaxalone, carisoprodol, methocarbamol). The method can be used to titrate the amount of treatment, e.g., the amount of drug to administer or the amount of intervention.

In one aspect, this disclosure features a method of providing anesthesia and monitoring subject for pain or analgesia during anesthesia. In certain embodiments, the method includes altering anesthesia according to the results of pain monitoring. In some embodiments, the method includes administering local anesthesia, regional anesthesia, general anesthesia, and procedural sedation. In some embodiments, the method includes administering anesthesia, e.g., intravenously, intramuscularly, intranasally, orally, and rectally. For example, the method includes administering, e.g., chloral hydrate, ketamine, meperidine, propofol, naloxone, morphine, flumazenil, nitrous oxide, halothane, isoflurane, lidocaine, bupivacaine, fentanyl and other opioids, halothane, enflurane, desfluorane, sevofluorane, barbiturates (thiopental, pentobarbital) and benzodiazepines (e.g., midazolam, diazepam, lorazepam).

In another aspect, the disclosure features a method of performing a medical decision, e.g., to evaluate the degree of pain in a subject who is unable to express a subjective assessment. For example, the subject is an infant, senile, unconscious, e.g., in a coma, e.g., an induced coma, a temporary coma, or a permanent coma. The method can include evaluating neural activity in the cortex, e.g., in SI, e.g., by detecting a hemodynamic parameter, e.g., blood oxygenation. The method can further include making a medical decision as a function of the detected neural activity. For example, the medical decision can be a decision to administer a pain reductant, e.g., an opiod.

In another aspect, the disclosure features a method for evaluating pain or analgesia in a subject, e.g., a mammal, e.g., a human. The method includes: evaluating activity in the primary somatosensory (SI) cortex of a subject by detecting a hemodynamic parameter, wherein activity in SI is indicative of a sensory state, e.g., pain sensation or numbness. For example, the hemodynamic parameter is a parameter assessing blood oxygenation or blood flow. The subject can be exposed to an external source of pain, e.g., before or during the evaluating, e.g., within 60, 30, 10, 5, 2, 1, 0.5, 0.1, or 0.01 minutes of the evaluating. In some embodiments, the subject experiences chronic pain (e.g., physiological, inflammatory, or neuropathic pain) or analgesia. The subject can be one who has sustained physical injury or surgery, or one who suffers from or is at risk of a disorder described herein.

The method can further include determining a baseline level of activity or other statistic of activity (e.g., average, minimum, maximum, frequency, amplitude). The method can detect changes in the hemodynamic parameter, e.g., using one or more light sources and one or more detectors. Information from the plurality of detectors can be used to localize the site of neural activity to a region within SI, e.g., to create a projection of neural activity in a map of the cortex. In some embodiments, the activity is compared to reference activity, e.g., activity in the same subject or a different subject, such as one or more reference subject. For example, a baseline for the subject, and the subject's baseline is compared to a corresponding reference baseline from the reference activity. Activity can be evaluated by comparing activity in one hemisphere to corresponding regions in the other hemisphere.

In certain embodiments, activity can be identified by determining a Bonferroni threshold and identifying regions with a p value of less than 10⁻², 10⁻³, 10⁻⁴, 10⁻⁵, or 10⁻⁶.

A single reference subject can be used to create reference information, as can a cohort of age, gender, and race matched subjects. Reference subjects can be, e.g., an individual who is or is not subjected to a pain stimulus, an individual who does or does not suffer from chronic pain, or an individual who does or does not suffer from analgesia, numbness. Where the reference activity is based on the same subject, the reference activity can be characterized by an alternative state, e.g., treated or not treated with an anesthetic or analgesic, relative to the current evaluation.

In certain embodiments, signal processing routines are used to process signals representative of the detected activity. For example, the hemodynamic parameter is corrected for a systemic physiological response.

In some embodiments, the detected activity is localized to one or more regions of the cortex. For example, activity in one or more Brodmann areas 2, 3a, 3b and 1 (e.g., all; or 3a, 3b, and 1; or 3b and 1; or 3a) are detected and distinguished from one another. Activity outside of the SI can also be evaluated, e.g., activity in the SII, e.g., area 40. In various embodiments, activity in one hemisphere is compared to activity in another hemisphere. Pain detected on one side of a subject is processed in the opposite hemisphere of the brain.

It is possible to monitor plasticity in the nervous system, e.g., in the CNS, e.g., by evaluating pain or other sensation by detecting cortical activity, e.g., using DOT. For example, plasticity that can be evaluated can include changes associated with amputation, peripheral nerve injury, spinal cord injury, other CNS nerve injury, burns, or trauma. Other plasticity include changes associated with learning, development, physical therapy, and rehabilitation.

In another aspect, the disclosure features a method of evaluating SI region function in the brain of a subject. The method includes: inducing pain in the subject; and evaluating neural activity in the SI region of the subject.

In another aspect, the disclosure features a method of evaluating SI region function in the brain of a subject who experiences chronic pain. The method includes: evaluating neural activity in the SI region of the subject who experiences chronic pain; and comparing neural activity in the evaluated subject to reference information. The reference information is corresponding neural activity for one or more normal subjects.

In still another aspect, the disclosure features a method of providing a treatment to a subject. The method includes: providing a treatment to the patient; evaluating the SI region of the patient for neural activity; and altering the treatment as a function of activity in the SI region that indicates pain. For example, the treatment includes administering a pain reductant (e.g., an opiod), an anesthetic or an anti-inflammatory agent. As another example, the treatment includes a physical stimulus, e.g., acupuncture. In an exemplary implementation, if the patient continues to experience pain, the amount or degree of treatment can be increased; if the patient experiences a reduction in pain, the amount or degree of treatment can be stabilized; if the patient experiences analgesia, the amount or degree of treatment can be reduced.

In another aspect, the disclosure features a method of providing anesthesia to a patient. The method includes: providing anesthesia to the patient; evaluating the SI region of the patient for activity; and, optionally, altering the amount of anesthesia, (e.g., increasing the amount of anesthesia) if activity in the SI region indicative of pain is detected.

In another aspect, the disclosure features a method of preparing a subject for neurosurgery. The method includes: having the subject perform a motor task or providing a sensory input to the subject; and evaluating hemodynamics in the cortex of the subject, thereby mapping a region of the cortex relative to the motor task or sensory input. The subject can be prepared, e.g., in a pre-operative condition. Information from the mapping can be used to direct surgery to the correct location.

In one aspect, the disclosure features a computer-readable database that includes a plurality of records. Each record includes: (a) one or more of the following: (i) information representing a subjective measure of pain or analgesia in a subject; and (ii) information indicating a pain relief treatment provided to the subject. The records can include (b) information representing SI activity in the subject.

In another aspect, the disclosure features a system for objectively evaluating pain or analgesia. The system can include one or more of: a light source, a detector, and a processor. The light source and the detector are configured for placement on a subject, e.g., on the scalp of a subject, such that the detector can evaluate hemodynamic parameters associated with the cortex.

The processor can be configured to evaluate signals from the detector for changes in hemodynamics as an indicator of neural activity in the cortex. The processor can store detected signals, e.g., as raw or processed information. For example, the processor can correlate signals with reference information to provide an objective measure of pain or analgesia based on activity in the cortex of a subject. The processor can be further configured to produce an image map based on information from the detectors. The processor can also include filters, e.g., filters to remove noise and to correct for a systemic physiological response.

In some embodiments, the system includes a plurality of light sources and a plurality of detectors. For example, the plurality of light sources are arranged in a first row on one hemisphere of the scalp and a second row on the other hemisphere of the scalp. The plurality of detectors can be arranged in four rows, one on either side of each row of light sources. A plurality of detectors can be arrange surrounding a light source, e.g., in an arc.

Information from an evaluation can be used to differentiate between symptoms of a neurological condition and an unrelated condition, e.g., one that masks an underlying neurological condition. An objective evaluation is useful for many reasons. It has obvious advantages in cases in which the subject cannot provide subjective information. The subject may be a non-human. The subject may be a human, but unable to communicate, e.g., due to unconsciousness, unwillingness, infancy, mental deficiency, damage to language centers, and psychiatric condition.

Other techniques can also be used to evaluate neuronal activity in humans. Examples of these techniques include magnetoencephalography (MEG), single proton emission tomography (SPECT), positron emission tomography (PET) and functional magnetic resonance imaging (fMRI), are now being used to discern CNS pathways involved in pain (Davis K D et al. Neuroreport. 1998 9:3019-23; Becerra LR et al. Magn Reson Med. 1999 41:1044-57; Casey KL Prog Brain Res. 2000 129:277-87.).

By “neurological condition” is meant a condition causing an alteration in normal or resting CNS (e.g., brain) activity either in the presence or absence of nervous system pathology. Non-pathological neurological conditions include, for example, psychiatric disorders, sensory stimulation (e.g., in response to painful, olfactory, gustatory, auditory, or visual stimuli), and cognition. Pathological neurological conditions include, for example, traumatic brain injury, neurodegenerative disease (e.g., Parkinson's disease, and Alzheimer's disease), and ischemic injury (e.g., caused by stroke).

By “psychiatric disorder” is meant a condition that affects mood or behavior. Exemplary psychiatric disorders include affective disorders (such as depression, bipolar disorder, mania, and dysphoria), anxiety disorders (such as anxiety and panic disorder), attention deficit hyperactivity disorder, psychotic disorders (such as schizophrenia and schizoaffective disorder), substance abuse disorders, phobias, and obsessive compulsive disorder.

By “cognition” is meant thinking skills that include language use, calculation, perception, memory, awareness, reasoning, judgment, learning, intellect, social skills, and imagination.

By “acute pain” is meant pain having a rapid onset and usually produced by direct stimuli such as trauma (e.g., resulting from acute injury or surgery) or burns. Typically, acute pain ceases when the stimulus is removed or the injured tissue has healed.

By “chronic pain” is meant persistent pain that is not caused by an acute stimulus. Most commonly, chronic pain results from a pathological condition such as infection, arthritis, chronic injury (e.g., sprain), cancer, and neurogenic pain (pain caused by nerve damage). An exemplary type of chronic pain is neuropathic pain.

By “indicative of” a condition is meant indicating the presence, absence, or extent of the condition.

By “neuropathic pain” is meant pain caused by peripheral nerve or central nervous system damage (e.g., stroke or spinal cord trauma). Neuropathic pain may include, without limitation, a burning sensation, hyperpathia, dysaethesia, allodynia, or phantom pain. Exemplary types of neuropathic pain include infective (e.g., post herpetic neuralgia and HIV neuropathy), metabolic (e.g., diabetic neuropathy and Fabry's disease), toxic (e.g., from lead or chemotherapy), traumatic/stretch injury (e.g., post incisional, trauma, phantom limb pain, and reflex sympathetic dystrophy/complex regional pain syndrome/causalgia), and idiopathic (e.g., trigeminal neuralgia/tic douloureux).

By “standard control image” is meant an image of CNS activity based on an average of control brain activity in a population, e.g., based on race, sex, socio-economic class, or geographic location. Control brain activity may be based on the presence or absence of a particular activity, e.g., sensation of pain.

By “therapeutic intervention” is meant a regimen intended to have a preventive, ameliorative, curative, or stabilizing effect. Examples of therapeutic interventions include pharmaceutical compositions (including candidate pharmaceutical compounds, pharmaceutical compounds not known to be effective in a particular neurological condition, and mixtures of compounds), physical stimuli (e.g., massage or acupuncture), electrical stimuli, thermal stimuli, electromagnetic radiation, or a surgical, medical, or dental procedure, or a combination thereof.

By “treating” is meant the medical management of a subject with the intent that a cure, amelioration, stabilization, or prevention of a neurological condition will result. This term includes active treatment, that is, treatment directed specifically toward improvement of a condition, and also includes causal treatment, that is, treatment directed toward removal of the cause of the condition. In addition, this term includes palliative treatment, that is, treatment designed for the relief of symptoms rather than the curing of the condition; preventive treatment, that is, treatment directed to prevention of the condition; and supportive treatment, that is, treatment employed to supplement another specific therapy directed toward the improvement of the condition. The term “treating” also includes symptomatic treatment, that is, treatment directed toward constitutional symptoms of the condition.

By a subject “undergoing” a medical, surgical, or dental procedure is meant a subject during substantive preparation for, execution of, or recovery from a medical, surgical, or dental procedure.

Other features and advantages of the invention will be apparent from the following description and the claims. Unless otherwise defined, all technical and scientific terms used herein have the same meaning as commonly understood by one of ordinary skill in the art to which this invention belongs. The disjunctive “or” also encompasses combinations of listed alternatives. Although methods and materials similar or equivalent to those described herein can be used in the practice or testing of the invention, suitable methods and materials are described below. All publications, patent applications, patents, and other references mentioned herein are incorporated by reference in their entirety. In the case of conflict, the present specification, including definitions, controls. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIG. 1A is graph showing a blood oxygenation level dependent (BOLD) signal during each thermal (46° C.) stimulus averaged for eight healthy volunteers. FIG. 1B is a statistical map of the region activated (green circle). T-statistics p<0.001.

FIG. 2A is a schematic representation of an experiment showing the damaged site (dark) over the dorsum of the lateral part of the left hand and a mirror map of the sensitive site in the right hand. FIGS. 2B-2C are images of the brain showing activation following a brush applied to the injured side in the right SI cortex (2B); there is no activation following a brush to the uninjured mirror image of the affected region (2C). FIG. 2D is a graph showing the percent signal change following application of 4 brush stimuli (dark bars above) as a function of time (sec).

FIG. 3A is a schematic representation of an experiment on cortical activation following a 45° C. stimulus in a patient with numbness involving the right V2 distribution. FIGS. 3B-3E show cortical images after heat stimulus applied to various portions of the face. An increase in activation follows stimulation of V3 ipsilateral to the region of numbness. The white box indicates the location of sensory loss, and the red box indicates the location of heat stimulus.

FIGS. 4A-4F are images of possible invasion of V2 into the region of V3 on the side of an anesthetic block. FIG. 4A shows locations of the slices. FIGS. 4B-4E show that activation of the left V2 (slice 12L) has no mirror correlation in the right V2 (12R) (bottom arrows—solid line, X) but has shifted to where the expected V3 activation should be (16R) (bottom arrows—dotted line, Y). The data is summarized in FIG. 4F.

FIGS. 5A-5C are a series of images illustrating activation of phantom hand by brush stimulation. FIG. 5A is a schematic illustration of the areas of the body studied. Activations representing the perceptual phantom limb activated by brush from concatenated runs to ipsilateral mouth, arm and foot areas (4+5+6) are shown in FIG. 5C. Activation from the contralateral side (1+2+3) (FIG. 5B) also is listed as a control. Two specific foci of activation in SI (Brodman area 2) and SII (area 40) were noted for the ipsilateral side and met the a priori threshold of p<2×10⁻⁴. At the Bonferroni threshold for all other brain regions (p<7×10⁻⁶), two other foci were noted in the supplemental motor area (area 6) and the posterior cingulate gyrus (area 24). On the unaffected side (runs 1+2+3), only one area of activation corresponding to SII and nearly mirroring the other SII activation, was observed at the a priori threshold.

FIG. 6A is a schematic representation of the optode arrangement for functional activation studies using DOT. FIG. 6B is a photograph of a DOT imager.

FIGS. 7A and 7B are graphs of differential absorption signals shown for a subject sitting and supine. FIG. 7C is a graph of the differential hemoglobin concentrations depicted during a motor task (black bar).

FIG. 8A is an image showing the optode arrangement for functional activation studies in a human subject. The measurements were taken simultaneously with fMRI measurements, and a surface profile from the subject's head was taken from anatomical MRI. The optode placements are indicated by the colored circles. FIG. 8B is a DOT image of absorption changes at 780 nm due to right motor task stimulation.

FIG. 9 is a schematic illustration for an exemplary optode geometry for covering the right and left motor cortex.

FIGS. 10A and 10B are graphs showing the results for deoxy-hemoglobin during left hand stimulation (A) and right hand stimulation (B), obtained by filtering the optical data with a band pass filter between 0.016-0.3 Hz and averaging 6 stimuli, for some of the source-detector couples. The thickness of the lines represents the standard error. The localized decrease in deoxy-hemoglobin is larger both in size and amplitude in the contralateral motor cortex (right for A and left for B).

FIG. 11A shows an arrangement of sources (squares) and detectors (circles) on the head of a subject (facing towards the top of the page). Oxyhemoglobin concentration (micromolar) is shown adjacent to each detector, and is depicted according to the graph shown in FIG. 11B. The stimuli was a temperature of 46° C. for two seconds on period average ISI of 12 seconds.

### DETAILED DESCRIPTION

We have developed, inter alia, an objective test for CNS activity, e.g., pain. Exemplary application include diagnosing the type, intensity, and presence of a neurological condition (such as a psychiatric disorder, e.g., depression or anxiety) or a therapeutic intervention (such as anesthesia or an analgesic compound). An objective determination of anesthesia is, for example, useful for surgery from both a diagnostic and clinical monitoring point of view. Preemptive anesthesia, that is provided in conjunction with an evaluation described herein, can be used to reduce or prevent incidence of pain as a result of surgical damage to nerves.

In certain embodiments, neural activity in the brain is evaluated by detecting a hemodynamic parameter, e.g., using DOT. The activity can be correlated with a neurological condition, e.g., pain. DOT is a technique that is capable of continuously monitoring and tomographically imaging changes in brain activation, e.g., by dynamically monitoring oxy- or deoxy-hemoglobin concentrations. By imaging a cortical region of a subject, DOT may be used to monitor the onset, duration, extent, and type of neurological condition (such as a psychiatric disorder) being experienced by the subject, or the effects of a therapeutic intervention like anesthesia or an analgesic.

In some embodiments, optical spectroscopy is used to assess the level of activation in one or more cortical regions of the brain, e.g., by determining blood flow or blood oxygenation in the region. Activation in cortical regions can also be evaluated by other techniques. Optical spectroscopy generally directs light into the cortex. The light can be from a single source. Then, the light scattered or emitted from the cortex is detected using one or more detectors. Increasing the number of sources and detectors increases the area of the cortex that can be monitored simultaneously. Information from monitoring the cortex, or specific portions thereof provides a determination of the presence, intensity, duration, and type of CNS activity experienced by a subject.

The monitoring can include methods that can resolve in spatial and temporal dimensions neural activity and identifying activated (or inactivated) regions of the cortex. Typically, this activation is identified by observation of a localized increased blood flow to an area of the cortex associated with one or more types of CNS activity, e.g., pain. By distinguishing between different areas of the cortex that respond to different types of CNS activity, the methods may be used to identify the type of CNS activity experienced by an individual, e.g., acute or chronic pain (e.g., chronic nociceptive pain (such as in arthritis) or neuropathic pain). Accordingly, the placement of optodes may be adjusted to monitor various portions of the cortex. One skilled in the art is able to provide the proper placement, spacing, and number of detector(s) and source(s) (one or more of each) based on the area of the cortex being monitored. For example, in the case of an average adult, the sources can be located between about 1.5 to 4 cm or 1.6 to 2.3 cm from one another, e.g., about 1.9 cm. Detectors can be located between about 2 to 4 cm or 2.6 to 3.4 cm from a source, e.g., about 3 cm. Sizing can be adjusted depending on the size of the subject.

The wavelength or wavelengths of light employed in the method will depend on the contrast agent used, e.g., hemoglobin, deoxyhemoglobin, or dye. Exemplary wavelengths include about 670 to 710 nm, e.g., about 690 nm, and about 810 to 850 nm, e.g., about 830 nm. Exemplary regions of the cortex that may be monitored include a region of a parietal lobe, frontal lobe, temporal lobe, or occipital lobe, e.g., a somatosensory cortex, motor cortex, or lateral prefrontal cortex. These regions may provide a direct measure of a particular CNS activity or may be indirectly related to such activity. For example, the motor cortex may be monitored for reflexive reactions to pain or painful stimuli. In addition, activity in cortical regions may be indicative of deeper brain activity related to the activity being monitored.

The level of activation of a cortical region may be determined from DOT using any method. For example, measurements from the cortex of a subject may be compared to various standards in order to determine a level of activation, e.g., as indicated by the localized blood flow. Standards include a reference measurement from the subject when not experiencing the CNS activity or when experiencing the CNS activity, a reference measurement from an inactivated or activated portion of the cortex (e.g., the motor cortex), or a reference from another subject, or aggregate of subjects, experiencing or not experiencing the activity (e.g., a standard control image). The regions of one or both hemispheres may be monitored, and a region in one hemisphere, activated or not, may serve as a reference for the other hemisphere.

Information from the detection, e.g., from DOT, can be processed, e.g., to projected onto a map of the brain, e.g., a 2D or 3D map. Information can be process, e.g., using high or low pass filters.

In one embodiment, signal processing routines are used to distinguish the neuronal response to pain (e.g., a pain stimulus) from a systemic physiological response. In some cases, a pain stimulus can cause blood pressure and heart rate changes, resulting in changes to systemic blood flow and blood volume. When detected by spectroscopy, e.g., near-infrared scpectroscopy (NIRS), such systemic changes are observed superimposed on the brain neuronal response. Accordingly, signal processing routines can be used to correct for systemic physiological changes to hemodynamics. One exemplary correction utilizes independent measurements of the heart rate and blood pressure in a data filter to distinguish the NIRS signal that arises from heart rate and blood pressure fluctuations from the NIRS signal that arises from the neuronal response to the pain stimulus. A second exemplary correction utilizes NIRS measurements from a large surface of the head. The systemic physiological fluctuations give rise to NIRS signals over the entire surface of the head while the neuronal response to the stimulus is localized to a small region over the somatosensory cortex. Spatio-temporal adaptive filters then are applied to distinguish the systemic signals from the neuronal signals.

The actual region of the cortex that is evaluated may vary depending on the particular use. In addition, an initial image or other dataform of a subject may be obtained to identify the location of a particular region of the cortex. Once identified, a smaller area encompassing all or a portion of the particular region may be monitored. For example, the location (or locations) of highest intensity may be monitored as a function of time.

Detection of CNS activity is useful in a variety of settings. First, it may be used to diagnose a neurological condition a subject is experiencing, e.g., pain (such as neuropathic, inflammatory, or acute pain), anesthesia (e.g., a reduction or lack of pain or other sensation either generalized or localized), or a psychiatric disorder (such as depression or anxiety). Such information is useful, for example, to determine a course of treatment. The detection of CNS activity that is indicative of a neurological condition, such as a psychiatric disorder, may be used to determine the need for additional medication, e.g., to aid in weaning addicts from addictive medications or to aid in increasing or otherwise changing medication to alleviate symptoms in a subject. The method may also be used to determine if a subject is appropriately anesthetized during a medical, surgical, or dental procedure, for example, to prevent or reduce acute or chronic pain. The methods may be further used to identify potential therapeutic interventions, e.g., therapeutic compounds, or to determine the effectiveness of an intervention.

In one exemplary method, DOT is first performed on a subject to determine a level of activation in a cortical region, e.g., the somatosensory cortex. The absolute level of activation may be indicative of a particular neurological condition. Alternatively, the level may be compared to a standard wherein an increased or decreased level of activation is indicative of a neurological condition, e.g., pain, in the subject. The relative change may be determined by comparison to various controls, for example, an image of the same subject when not experiencing the neurological condition or a standard control image. The level of activation may also be used to determine the extent of the neurological condition, and the location of activation may be used to determine the type of condition experienced, e.g., acute pain or neuropathic pain. This method may be especially useful during medical, surgical, or dental procedures to ensure that appropriate anesthesia has been provided to the subject. Additionally, as stated above, the method may be used to determine whether a subject is in actual need of a therapeutic intervention or a change in intervention.

In another example, cortical activity can be evaluated to identify potential therapeutic interventions, e.g., analgesic compounds. This method includes performing DOT on a subject in the presence of a stimulus following administration of a test intervention (e.g., a test compound) to determine the level of activation of a cortical region, e.g., by producing an image of the region. In this example, the level of activation is indicative of the effectiveness of the test intervention. A test intervention that reduces the level of activation can be used as a therapeutic, e.g., to reduce responsiveness, e.g., as an analgesic. The stimulus can be designed to cause a change (increase or decrease) in CNS activity indicative of a neurological condition. The stimulus may be noxious or non-noxious depending on the type of activity being evaluated. For example, visual, gustatory, auditory, olfactory, or tactile stimuli may be employed. In addition, chemical, thermal, or electrical stimuli may also be used. In one example, a thermal stimulus is used to induce pain in a subject. In another example, an object representing a soiled item may be used as a stimulus for subjects suffering from obsessive compulsive disorder (Breiter HC et al. Arch Gen Psychiatry. 1996 53:595-606.). The level may be determined relative to controls as described herein.

Any intervention may be employed in this method, for example, pharmaceuticals, such as organic compounds, inorganic compounds, peptides or proteins, and nucleic acids (e.g., gene products). Additional interventions include physical stimulation (e.g., via massage or acupuncture), thermal stimulation (warm or cold), electromagnetic radiation, electrical stimulation, or a surgical, medical, or dental procedure. The method may also be used to determine the length of time that a particular intervention is effective by periodically monitoring activation as a function of time, e.g., time after intervention. Moreover, the method may be used to determine the effect of intervention on chronic neurological conditions, e.g., pain, by measuring the effect of the intervention on the condition experienced by a subject in the absence of any external stimulus. This method may also be used to quantify the effects of a particular intervention, e.g., to determine proper dosing or frequency and possible synergistic or detrimental interactions with other interventions.

In an alternative embodiment, potential therapeutic interventions may be evaluated by testing on subjects suffering from a neurological condition, e.g., chronic pain. In this embodiment, DOT is used to monitor the level of activation indicative of the neurological condition after the administration of a test intervention. The change in the level of activation is then indicative of the effectiveness of the test intervention.

In addition, the methods described herein may be used to correlate subcortical brain activity with cortical activity. In this method, subcortical activity is measured by a technique such as fMRI, MEG, SPECT, or PET, and cortical activity is measured by DOT. By comparing the activation patterns of one or more subcortical regions with one or more cortical regions, it is possible to correlate deeper brain function with cortical activation. Once a deeper brain function is correlated with a cortical activity, the cortical activity can be monitored by the methods described herein.

A variety of neurological conditions including, but not limited to, pain, anesthesia, psychiatric disorders (such as depression and anxiety), sleep states, and motor disorders such as Parkinson's disease may be evaluated by examination of cortical activation in the manner as described herein.

### Diffuse Optical Tomography (DOT)

DOT refers to various noninvasive methods of imaging different tissues of a body or organ. (See, e.g., Strangman et al. (2002) Biol. Psychiatry, 52:679-693; Jasdzewski et la. (2003) Neurolmage 20:479-488). Generally, DOT relies on the emission of light from a light source into the body, then detecting the light scattered from various tissues of the body. For example, since light scattered by hemoglobin in blood differs from light scattered by other tissues, DOT has been applied to the imaging of blood within the body. In addition, because the absorption of light by deoxyhemoglobin is different from the absorption of light by oxyhemoglobin, DOT has been used to locate areas of high or low oxygenation in the body by determining decreases or increases in the intensity of scattered light. DOT affords continuous, in vivo, real time measurements of cerebral oxy-hemoglobin (HbO₂), deoxy-hemoglobin (Hb), and total hemoglobin (HbT=HbO₂+Hb) up to several centimeters in depth (Villringer A et al. Trends Neurosci. 1997 20:435-42; Benaron DA et al. J Cereb Blood Flow Metab. 2000 20:469-77.). Blood flow may also be determined through bolus measurements of intrinsic or extrinsic contrast agents. Using these hemoglobin fractions as contrast agents, optical spectroscopy can provide crucial information on cerebral hemodynamics and oxygenation during different acute and chronic brain conditions, as well as detect regions of brain ischemia, necrosis, and hemorrhage (Kuebler WM et al. J Cereb Blood Flow Metab. 1998 18:445-56; Patel J et al. Pediatr Res. 1998 43:34-9.). DOT can include a single (or few) source-detector pair optical spectroscopy (or near infrared spectroscopy, NIRS) measurements. For example, multiple sources can be used. A source can be evaluated using one or more detectors. For example, it has been demonstrated in animals and humans that NIRS is capable of detecting temporal changes in cerebral hemodynamics and oxygenation under various physiological and pathophysiological conditions, as well as intracranial hematomas having several times higher absorption than the surrounding brain tissue (Gopinath SP et al. J. Neurosurg. 1995 83:438-44; Villringer A et al. Trends Neurosci. 1997 20:435-42.).

By combining several light sources and detector pair measurements, optical spectroscopy can be used to image the spatial variations of absorption and scattering properties of highly scattering brain tissue, and provide both functional and low resolution anatomical images. Development of continuous-wave and frequency-domain optical instruments provided further sophistication, and development of imaging instrumentation, algorithms, software, and optical interfaces lead to improved spatial and temporal resolution. The noninvasive nature, low cost, and capability to obtain continuous, real-time information on cerebral hemodynamics and oxygenation under various physiological and pathophysiological conditions of DOT represents its major advantage over other techniques such as CT and MRI. An exemplary DOT system and parameters for observation of the cortex is described in U.S. Pat. Nos. 6,516,214 and 6,577,884.

An exemplary DOT instrument is as follows. The multi-channel continuous wave (CW) optical imager has eighteen lasers (intensities driven at 18 different frequencies) and sixteen detectors. The eighteen lasers are divided into nine lasers at 690 nm and nine at 830 nm. A master clock generates the eighteen distinct frequencies between 4 kHz and 7.4 kHz in approximately 200 Hz steps. These frequencies are then used to drive the individual lasers with current stabilized square-wave modulation. The detectors are avalanche photodiodes (APDs, Hamamatsu C5460-01). Following each APD module is a bandpass filter, a cut-on frequency of ˜500 Hz to reduce 1/f noise and the 60 Hz room light signal, and a cut-off frequency of ˜16 kHz to reduce the third harmonics of the square-wave signals. After the bandpass filter is a programmable gain stage to match the signal levels with the acquisition level on the analog-to-digital converter within the computer. Each detector is digitized at ˜40 kHz. The individual source signals are then obtained by use of a digital bandpass filter, for example, a discrete Fourier transform or an infinite-impulse-response filter. The individual source signals are separated off-line by using an infinite-impulse-response filter with a 10-50 Hz band pass frequency, enabling the production of optical images with a frame rate >10 Hz. The system may also be expanded to thirty-two lasers and thirty-two detectors. In certain implementations, it is also possible to use one or two lasers to provide light for multiple different sources located on the subject.

Contrast agents other than hemoglobin may be used in DOT. In this embodiment, a dye or other compound that is capable of absorbing the source wavelength of light, is introduced into the blood of a subject. As the amount of the dye present in a particular portion of the brain will depend on the blood flow, the amount of dye detected by DOT may be used to determine the local hemodynamics of a region of the body. Exemplary contrast agents are non-toxic, and examples of contrast agents include, without limitation, rifampin (red), β-carotene (orange), tetracycline (yellow), indocyanine green, Evan's blue, methylene blue; soluble inorganic salts such as copper sulfate (green or blue), Cu(NH₃)₄²⁺ (dark blue), permanganate (purple), NiCl₂ (green), CrO₄²⁻ (yellow), Cr₂O₇²⁻ (orange); proteins such as rhodopsin (purple and yellow forms) and green fluorescent protein (fluoresces green under blue light); and any of the Food and Drug Administration (FDA) approved dyes used commonly in foods, pharmaceutical preparations, medical devices, or cosmetics, such as the well-characterized non-toxic sodium salts FD&C Blue No. 1 (Brilliant Blue FCF), FD&C Green No. 3 (Fast Green FCF), FD&C Red No. 3 (Erythrosine), FD&C Red No. 40, FD&C Yellow No. 5 (Tartrazine), and FD&C Yellow No. 6 (Sunset Yellow FCF).

The dispersible chromophores listed above are generally (1) water-soluble at physiological pH, although fat-soluble chromophores (such as β-carotene) will also work if they are rapidly flushed from tissue, or (2) digestible or metabolizable through enzymatic pathways (such as methylene blue, which is rapidly metabolized by mitochondrial reductases, and proteins which are digested by proteases). In some cases, it may be possible to modify a chromophore to improve its dispersibility. A particular advantage of protein chromophores is that they can be conjugated to degradation inducing moieties, such as degradation signaling polypeptides using standard biochemical techniques. For example, green fluorescent protein can be conjugated to ubiquitin, which facilitates breakdown of the protein into small, invisible peptides by the eukaryotic ubiquitin proteolysis pathway.

Evaluating cortical activation, e.g., using DOT, other spectroscopy (e.g., near infrared spectroscopy) or other assessment of a hemodynamic parameter, can be used as an inexpensive and objective index of a CNS condition, e.g., pain. Exemplary applications include the following.

Anesthetic Blockade of Afferent Pain Fibers. Surgery frequently causes damage to the peripheral nervous system which results in chronic pain or dysesthesias in an estimated 20-50% of subjects (Dajczman E et al. Chest. 1991 99:270-4; Wallace et al., 1996; Rogers ML et al. Eur J Cardiothorac Surg. 2000 18:711-6.). The damage is thought to occur as a result of afferent barrage of the nervous system by unblocked C fiber activity. Current anesthetic techniques do not measure whether the blockade is complete. For example, although an epidural blockade affects pin-prick (commonly tested in the OR), it does not provide total afferent somatic blockade during cholecytectomy as assessed by somatosensory evoked potentials (Dahl JB et al. Reg Anesth. 1990 15:73-5; Dahl JB et al. Br J. Anaesth. 1992 69:117-21; Dahl JB et al. Acta Anaesthesiol Scand. 1992 36:378-82.). These changes correlate with other “stress indices” such as plasma cortisol and glucose. Furthermore, multimodal drug combination has no measurable long-term advantage (Doyle E et al. Br J. Anaesth. 1998 80:147-51; Rosaeg OP et al. Reg Anesth Pain Med. 2001 26:125-30.). An objective measure of afferent activation of known cortical regions as an index of anesthetic blockade can be used as a basis to evaluate the efficacy of pre-emptive analgesic methods. Since millions of surgeries are performed in the United States every year, this would be of enormous benefit by reducing chronic pain or dysesthesia, e.g., associated with surgery.

Moreover, many current methods for measuring anesthesia depth do not specifically measure analgesia. In addition there are a number of reports on complications relating to awareness under anesthesia. Awareness remains a serious complication of general anesthesia with potential adverse psychological sequelae. Even during seemingly adequate general anesthesia, implicit memory may be retained along with the ability to subconsciously process auditory stimuli. As a result behavior may be modified and postoperative progress influenced. Accordingly, evaluating pain and analgesia of a patient by objective means, e.g., by DOT or other method described herein, can be used to modify surgical procedure, anesthesia administration, or other medical treatment of a patient. Typically the patient is sedated or unconscious, e.g., as a result of anesthesia.

Rehabilitation. Injury, stimulation, and training can induce changes in the homuncular organization of the primary somatosensory (SI) (Flor H MMW Fortschr Med. 1999 141:16.). Phantom limb pain (a forme fruste example of pain following damage to the peripheral nervous system) has been identified as a perceptual correlate of this cortical reorganization. Previous work, using functional magnetic resonance imaging (fMRI) and magnetoencephalography, has demonstrated a correlation of an abnormal cortical representation in patients with pain and a normal representation in patients without phantom pain (Flor H et al. Nature. 1995 375:482-4.). In addition, the use of physical therapy methods such as myoelectric prostheses in amputees results in reduced phantom limb pain. This finding correlates with the neuroimaging results of reduced cortical reorganization. Cortical activity in non-painful phantom limb sensations exhibits specific activity in SI and posterior parietal cortex and decreased activations in the ipsilateral SII cortex. The efficacy of analgesics such as opioids may be evaluated by demonstrating a correlation of pain reduction concomitant with a reduction in cortical reorganization using a method described herein, e.g., using DOT or other method to detect cortical activity. Thus, evaluating cortical somatotopic plasticity in patients can be used as an objective measure of therapeutic efficacy.

Evaluation of Pain in an Outpatient Setting. The methods described herein, e.g., using DOT or other method to detect cortical activity can be used to evaluate pain in the emergency room and in the chronic pain clinic setting where evoked pain is part of the clinical evaluation (e.g., light brush producing pain in the distribution of a damaged nerve—allodynia). In one embodiment, the method can be used as an objective basis for the evaluation of drugs, for example, in the outpatient setting, in drug trials, and so forth.

Evaluation of neural activity in the cortical region can be supplemented by information from other evaluation methods, including, e.g., patient verbal reports (Mantha S et al. Anesth Analg. 1993 77:1041-7.); neurological evaluation (Serra J Acta Neurol Scand Suppl. 1999 173:7-11; 48-52.); quantitative sensory testing (Zaslansky Ret al. J Neurol Sci. 1998 153:215-38; Greenspan JD Curr Pain Headache Rep. 2001 5:107-13.); electrophysiological monitoring (EMG, MEG (Kagiki R et al. Prog Neurobiol. 2000 61:495-523)); single fiber recording (LaMotte RH et al. J. Neurophysiol. 1984 51:1434-50; Schmidt R et al. J. Neurophysiol. 1997 78:2641-8; Olausson B Exp Brain Res. 1998 122:55-61.); cellular recordings from the human brain; and skin punch biopsies (Holland NR et al. Neurology. 1997 48:708-11.); and other tomographic information of the brain.

### An Exemplary System

An exemplary integrated system can be used to detect neural activity in the cortex and store the information in a database, e.g., in association with other information about the subject. The system can include a light source and one or more detectors, e.g., DOT instrumentation. The system can include a database server that is configured to store records, e.g., as a datastructure. For example, a datastructure can be used that includes a first field containing or pointing to clinical or other personal information about a subject (e.g., name, identifier, gender, and so forth) of the individual and a second field containing or pointing to information about cortical activity, e.g., under one or more conditions. For example, the information about coritical activity can be raw or processed data obtained by a method herein, e.g., DOT. For example, the record can associate information from the same subject under treated and untreated conditions, e.g., treated with an analgesic or anesthetic.

The methods and other features described herein can be implemented in digital electronic circuitry, or in computer hardware, firmware, software, or in combinations thereof. Methods can be implemented using a computer program product tangibly embodied in a machine-readable storage device for execution by a programmable processor; and method actions can be performed by a programmable processor executing a program of instructions to perform functions by operating on input data and generating output. For example, methods can be implemented advantageously in one or more computer programs that are executable on a programmable system including at least one programmable processor coupled to receive data and instructions from, and to transmit data and instructions to, a data storage system, at least one input device, and at least one output device. Each computer program can be implemented in a high-level procedural or object oriented programming language, or in assembly or machine language if desired; and in any case, the language can be a compiled or interpreted language. Suitable processors include, by way of example, both general and special purpose microprocessors. A processor can receive instructions and data from a read-only memory and/or a random access memory.

Generally, a computer will include one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including, by way of example, semiconductor memory devices, such as EPROM, EEPROM, and flash memory devices; magnetic disks such as, internal hard disks and removable disks; magneto-optical disks; and CD-ROM disks. Any of the foregoing can be supplemented by, or incorporated in, application-specific integrated circuits.

Data structures, trees, databases, and other information formats described herein can be stored in a machine accessible memory (e.g., volatile or non-volatile memory, within a CPU or external to a CPU) or on machine-readable medium (e.g., a hard disk, CD-ROM, and so forth.

The system includes a processor, a random access memory (RAM), a program memory (for example, a writable read-only memory (ROM) such as a flash ROM), a hard drive controller, and an input/output (LO) controller coupled by a processor (CPU) bus. The system can be preprogrammed, in ROM, for example, or it can be programmed (and reprogrammed) by loading a program from another source (for example, from a floppy disk, a CD-ROM, or another computer).

The hard drive controller is coupled to a hard disk suitable for storing executable computer programs, including programs for evaluating information obtained from a subject (e.g., comparing neural activity in a subject with reference information), and data including storage. The I/O controller is coupled by means of an I/O bus to an I/O interface. The I/O interface receives and transmits data in analog or digital form over communication links such as a serial link, local area network, wireless link, and parallel link.

One non-limiting example of an execution environment includes computers running Linux Red Hat OS, Windows XP (Microsoft), Windows NT 4.0 (Microsoft) or better or Solaris 2.6 or better (Sun Microsystems) operating systems. Browsers can be Microsoft Internet Explorer version 5.0 or greater or Firefox version 1.5 or greater. Other environments can also be used.

Cortical Responses to Tactile and Skin-heating Stimuli: Differentiating Pain from Tactile Stimuli

Anatomical, neurophysiological, and imaging data confirm a role of SI cortex in pain processing. In anesthetized monkeys, optical signal imaging and extracellular electrode recording methods show different regions of the somatosensory cortex are activated, and the thermal heating showed temporal summation. The magnitude of the intrinsic signal correlated with increasing temperatures (Tommerdahl M et al. J. Neurophysiol. 1996 75:2662-70.). Thus, repetitive (summation) stimuli are expected to produce a clear signal in the cortex. Using wave-form based analysis, we have observed that a consistent activation in the SI cortex is observed across individuals. Likewise, it is possible to evaluate SI activation in various subjects and under various conditions, e.g., to compare different forms of pain, e.g., physiological pain, inflammatory pain, and neuropathic pain.

Loss of SI cortex in both humans and non-human primates is associated with selective loss of cutaneous pain sensibility (Russell WR Brain 1945 68:79-97; Kenshalo DR et al. J. Neurophysiol. 1989 62:429-36.). The traditional view of the role of SI in pain has been called into question even though there are a few neurons in Brodmann areas 3b and 1 of the somatosensory cortex that respond specifically to noxious stimuli (Kenshalo DR et al. J. Neurophysiol. 1983 50:1479-96.). Current evidence is that there is dual projection of neurons to SI, to the upper and middle layers of SI. Damage to the parietal cortex that spares 3b, may result in hyperalgesia. In addition, the SI cortex may respond differentially to non-noxious and noxious stimuli to the same skin site: non noxious stimuli activates areas 3b and 1, and noxious stimuli only activate 3a of the primary somatosensory cortex (Thammerdahl et al., 1996; Whitsel BL et al. Pain Imaging IASP Press:Seattle 2000, pp. 47-93.). This differentiation has at least two implications. The first is that clear differences using optical imaging in nociceptive processing vs. non-noxious sensory processing can be differentiated (e.g., only area 3a will be observed as activated following noxious stimuli). However, in chronic pain patients, because of differences in sensitization effects (e.g., repetitive stimuli, brush-evoked allodynia) a combination of SI activations (e.g., 1, 3a, and 3b) will be observed. These regions can be differentiated using fMRI and optical imaging, allowing one to define differences in pain vs. non-painful stimuli. This differentiation has application for evaluation and development of use in a number of clinical conditions including: (a) chronic pain; (b) pain under interventional conditions (e.g., surgery); and (c) therapeutic conditions (e.g., rehabilitation methods or pharmaceutical administration).

### Functional Imaging Data

### Primary Somatosensory (SI) Activation by a Painful Stimulus

The primary somatosensory cortex has been implicated in pain processing based on anatomical, electrophysiological, and lesion studies. Human brain-imaging studies do not consistently demonstrate pain-related activation of SI. However, the bulk of evidence now points to human imaging studies demonstrating a defined role of the SI region in sensory aspects of pain, including localization and discrimination of pain intensity. We have recently demonstrated that this can be shown in individuals if the temporal nature of the blood oxygenation level dependent (BOLD) response is taken into account.

Cortical Activation in a Subject with Allodynia

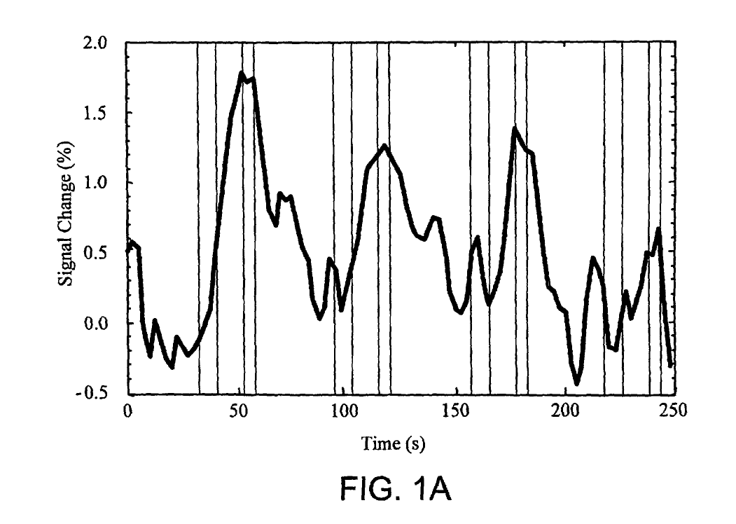

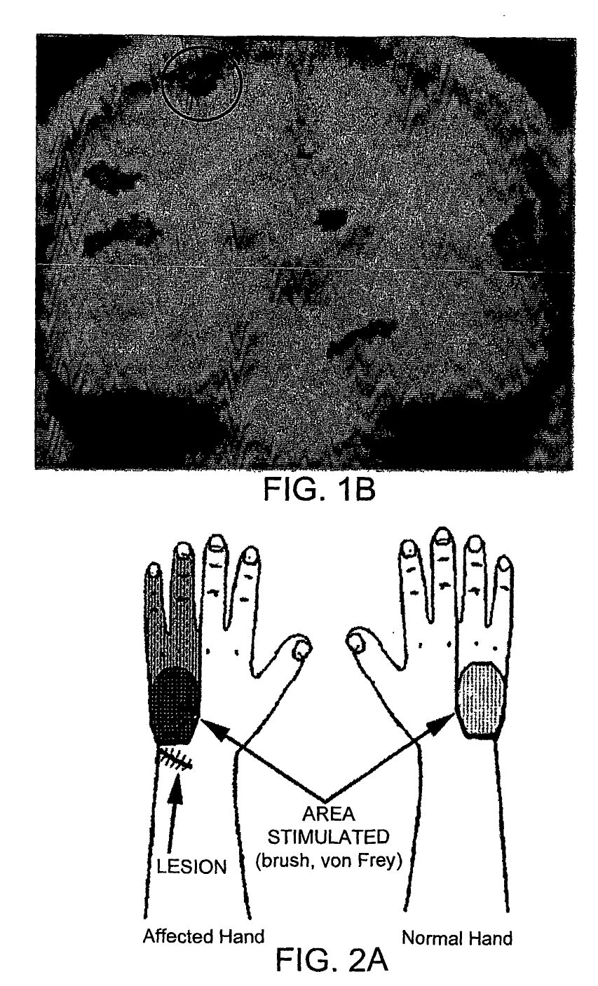

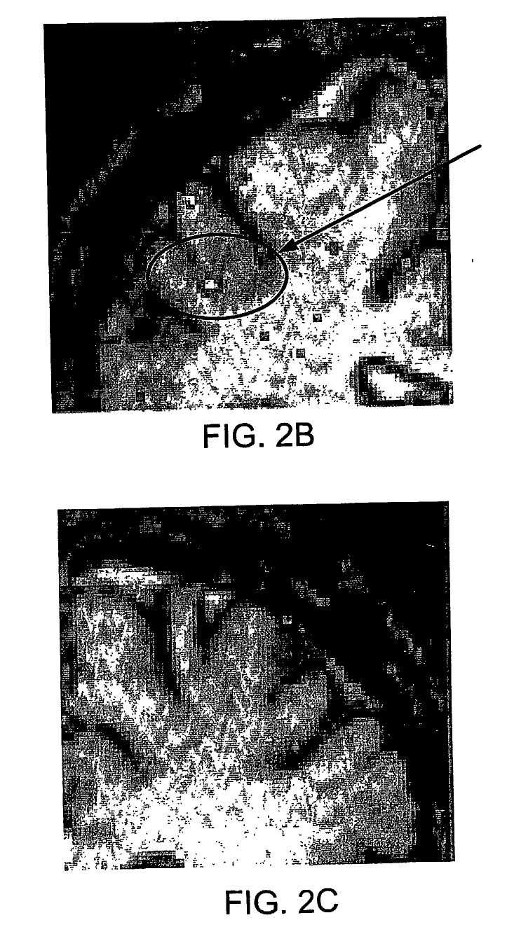

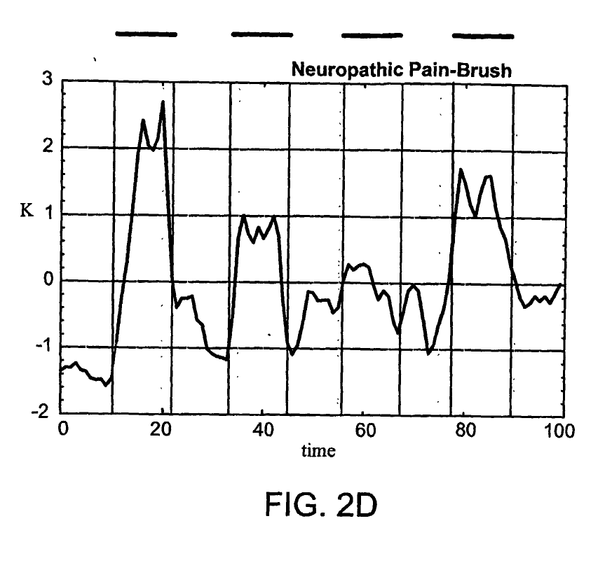

We have recently demonstrated that activation in the SI cortex can be reliably detected following noxious heat at a group and an individual level. Seven of eight subjects showed robust activation in SI (FIGS. 1A and 1B). Following brush induced allodynia, we also observed SI activation (FIGS. 2A-2D). The data suggest that the SI is involved in discrimination of the pain (e.g., site and intensity).

Cortical Plasticity following Sensory Loss

### Neurosensory Deficit in a Patient

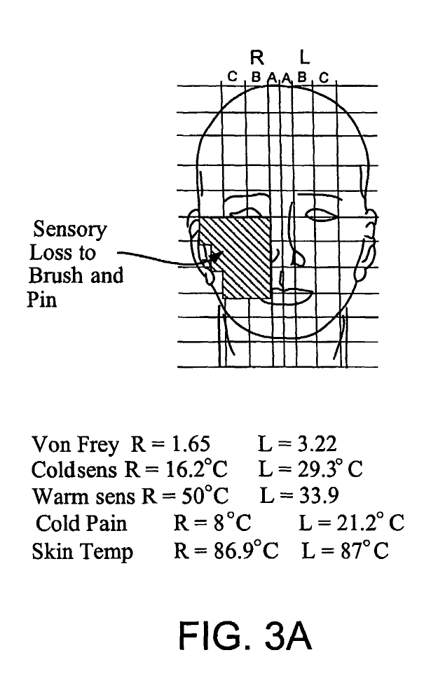

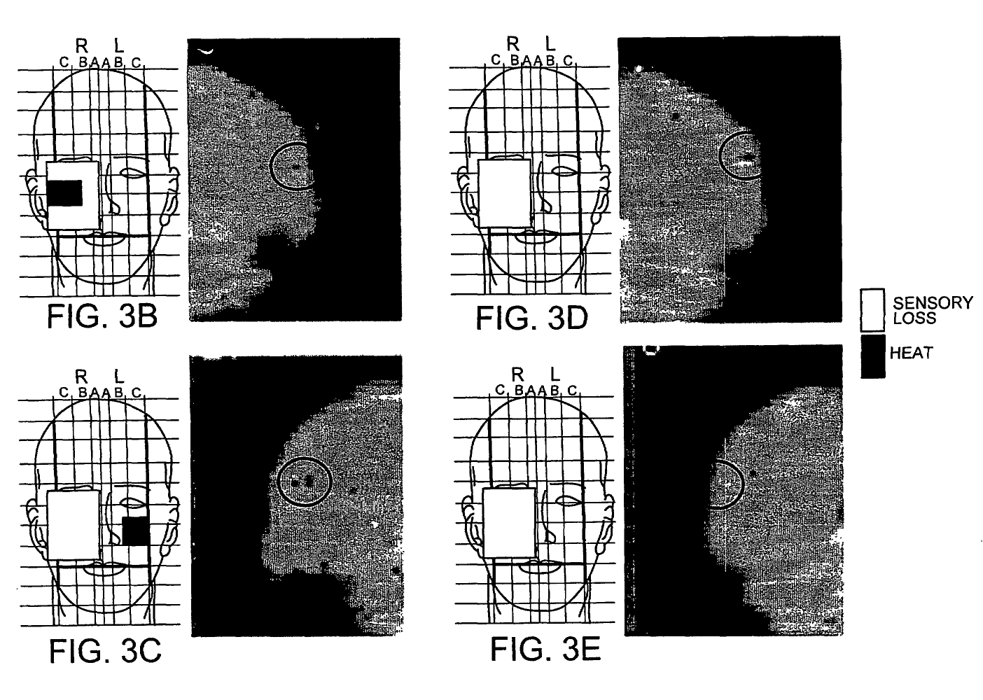

In a similar manner as that described above for a healthy volunteer, we examined fMRI activation in a female patient who had facial burning and numbness following trigeminal rhizotomy one year previously for trigeminal neuralgia. Medications, including gabapentin and tegretol, had been unhelpful. Careful questioning and psychophysical evaluation was performed. Sensory testing to brush and pin were significantly diminished in the right V2 distribution including the upper lip. Von Frey testing showed a decrease on the right (3.22 hair) vs. the left side of the face (1.65 hair). She also had decreased warm and cold sensation on the affected side vs. the intact side. She underwent functional neuroimaging for thermal stimuli applied to the V2 and V3 distributions on the right and left. FIGS. 3A-3E show the results of cortical activation from this patient. There was a decrease in activation to a 45° C. stimulus applied to the affected area and an apparent increase in signal for the same stimulus applied to the ipsilateral V3 distribution.

### Local Anesthetic Blockade in Healthy Volunteers

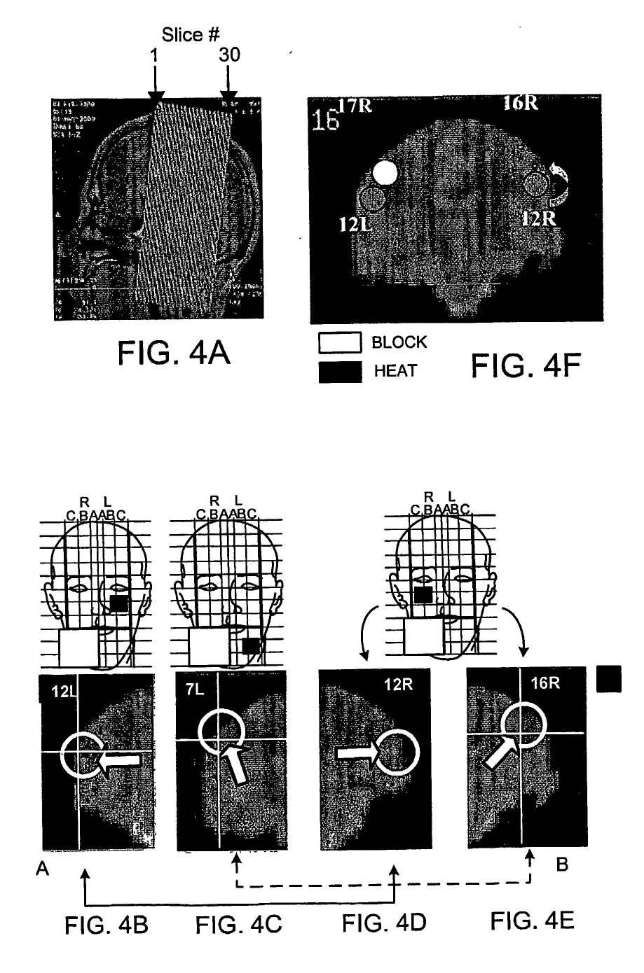

We have examined CNS activation in two subjects following inferior alveolar nerve blockade (V3) with local anesthetic (FIGS. 4A-4F). Following stimulation of the left V3, activation was observed in the ipsilateral spV and contralateral thalamus and cortex. However, following stimulation of the V2 area on the right, activation shifted into the region of the cortex that was observed to activate in the mirror V3 cortical region. Such invasion into “missing” territories has been observed in animals and humans (FIGS. 4A-4F).

### Cortical Representation of the Phantom Hand

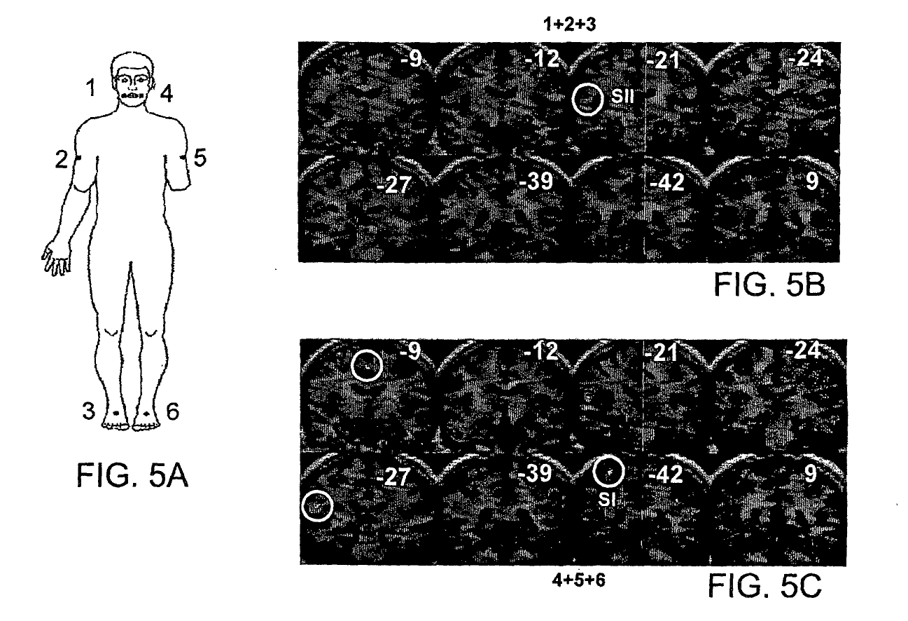

We studied a patient after amputation of an arm and found that in less than 24 hours, stimuli applied on the ipsilateral face were referred in a precise, topographically organized, modality-specific manner to distinct points on the phantom limb (FIGS. 5A-5C). Functional magnetic resonance imaging (fMRI), performed one month later, showed that brush-evoked activity in the brain demonstrates objective signal changes which correlate with perceptual changes in the phantom hand. This finding in humans corresponds to the findings of immediate plasticity in cortical pathways described in animals, including primates. The results suggest that massive reorganization of sensory pathways occurs very soon after amputation in humans, and the reorganization is due to activation of ordinarily silent inputs rather than sprouting of new axon terminals.

Cortical Responses to Morphine: Differences from SI Activation by Pain

We have examined, using functional magnetic resonance imaging (fMRI), the response of morphine in opioid naïve individuals and have demonstrated activation in deep subcortical structures including those involved in reward. Some of these have an oppositely valenced signal to that observed for aversive painful stimuli. In the SI cortex, we observe a decrease in signal in SI following low-dose morphine in a similar manner to that observed for general anesthesia (see below). This observation is in counter distinction to that observed for cortical activation by painful stimuli (see above).

### Cortical Responses to General Anesthesia

We also investigated the effects of the general anesthetic agent propofol on cerebral structures involved in the processing of vibrotactile information. Using positron emission tomography (PET) and the H(2)(15)O bolus technique, we measured regional distribution of cerebral blood flow (CBF) in eight healthy human volunteers. They were scanned under five different levels of propofol anesthesia. Using a computer-controlled infusion, the following plasma levels of propofol were targeted: Level W (Waking, 0 microg/ml), Level 1 (0.5 microg/ml), Level 2 (1.5 microg/ml), Level 3 (3.5 microg/ml), and Level R (Recovery). At each level of anesthesia, two 3-min scans were acquired with vibrotactile stimulation of the right forearm either on or off. The level of consciousness was evaluated before each scan by the response of the subject to a verbal command. At Level W, all volunteers were fully awake. They reported being slightly drowsy at Level 1, they had a slurred speech and slow response at Level 2, and they were not responding at all at Level 3. The following variations in regional CBF (rCBF) were observed. During the waking state (Level W), vibrotactile stimulation induced a significant rCBF increase in the left thalamus and in several cortical regions, including the left primary somatosensory cortex and the left and right secondary somatosensory cortex. During anesthesia, propofol reduced in a dose-dependent manner rCBF in the thalamus as well as in a number of visual, parietal, and prefrontal cortical regions. At Level 1 through 3, propofol also suppressed vibration-induced increases in rCBF in the primary and secondary somatosensory cortex, whereas the thalamic rCBF response was abolished only at Level 3, when volunteers lost consciousness. We conclude that propofol interferes with the processing of vibrotactile information first at the level of the cortex before attenuating its transfer through the thalamus.

Combining fMRI and DOT Simultaneously to Demonstrate Anatomic and Physiological Correlations

Instrument characteristics. Using an inexpensive, portable and flexible DOT system, bedside monitoring of changes in brain hemoglobin were made. A system having two or more source wavelengths (to allow determination of both Hb and HbO₂), multiple detectors, high data acquisition rate capabilities, high sensitivity, individualized detector gain, and the ability to simultaneously record stimulus signals for guaranteed time-locking with the optical signal was employed.

fMRI and DOT. To demonstrate the utility of the instrument for non-invasive use on humans, we conducted simple experiments with young, healthy volunteers. These results demonstrate the ability of a fast CW imaging system to produce reliable, accurate measures of changes in hemoglobin concentrations. A basic motor protocol was chosen to investigate the recordability of optical signals from the brain, and to allow comparison with validated findings of deoxy-hemoglobin and volume changes found in fMRI studies.

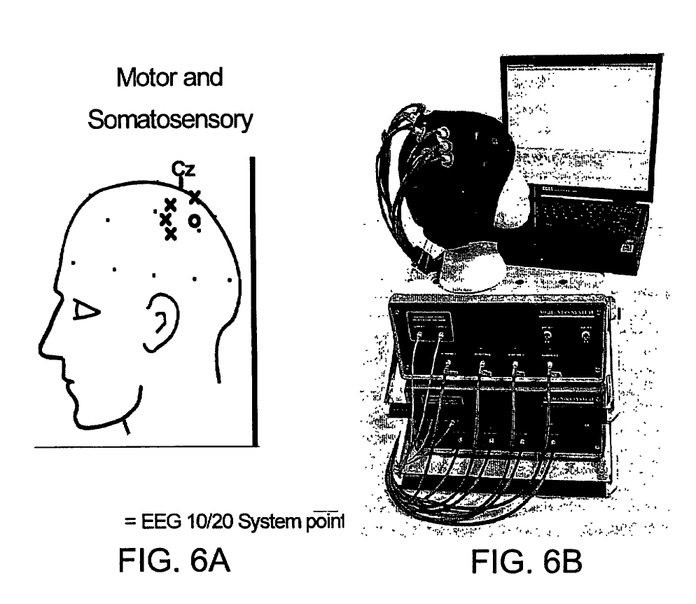

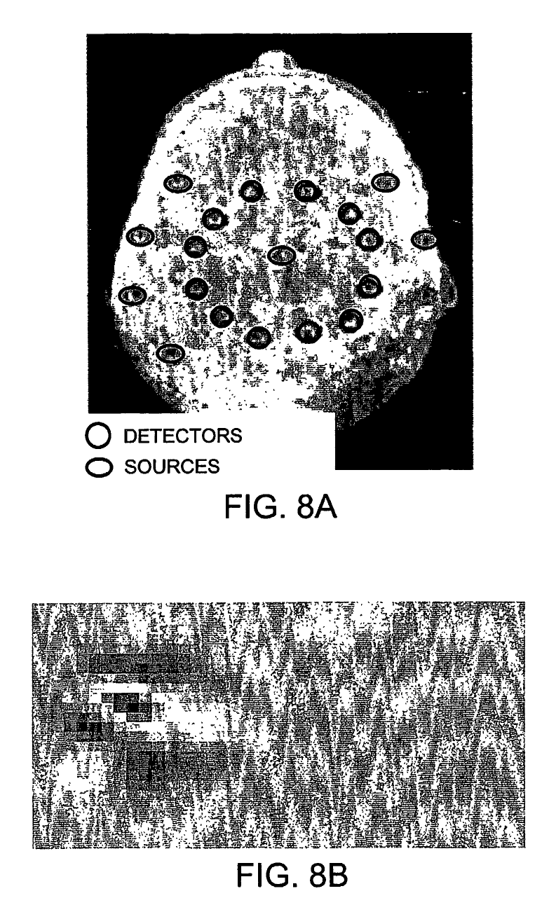

The positions of the optodes were separately determined for each paradigm so as to cover an area over the cortex that was expected to be activated according to prior fMRI studies (Kwong KK et al. Proc Natl Acad Sci USA. 1992 89:5675-9; Belliveau JW et al. Science. 1991 254:716-9; Bandettini P A et al. Magn Reson Med. 1993 30:161-73.). FIG. 6A depicts the optode placement relative to the international 10-20 system (Jasper, HH Clin Neurophysiol. 1958 10:371-375; Klem GH et al. Electroencephalogr Clin Neurophysiol Suppl. 1999 52:3-6.). FIG. 8A shows the optode placement for functional studies. FIG. 8B shows an exemplary image obtained using DOT.

To couple the fibers to the head, we used a flexible plastic spine as the substrate to which we attached the fibers. Side-firing fiber bundles (3 mm core diameter, Fiber Optic Technologies) were developed to maintain a low profile on the scalp and to minimize motion artifact. The side-firing fibers may be custom made from a bundle of glass fibers that are mounted inside of an SMA connector on the end that couples to the sources and detectors to the instrument. At the other end, the bundle of fibers is bent 90 degrees in a small radius and held in position with an epoxy resin. This allows the fibers to lie flat on the subject of the head with the light entering and exiting from the side of the fibers to make optimal contact with the subject's scalp. FIG. 6B shows an image of the setup with the cap attached to a head-phantom.

At the start of the experiment, the subject remained seated in a chair while the cap was put on. No gel or special hair treatment was required except to wiggle the fibers through the hairs to improve the contact with the skin. The first subject was simply asked to close his eyes and relax during two 40 sec recording periods. For the first such period, the subject sat upright in a chair and for the second period, the subject was asked to lay supine. The purpose of these two recordings was to see what physiological variables could be recorded by the instrument in a simple resting baseline condition. Another subject sat upright and was asked to perform a four-finger flexion/extension task. The task was to flex and extend the four fingers of a designated hand as quickly as possible for 15 sec blocks, alternated with 15 sec periods of rest, as designated by visual stimuli on a computer monitor. Signals were recorded continuously for 315 sec runs (10 active periods, 11 resting periods) from the region surrounding C3 and C4 (international 10-20 system designations).

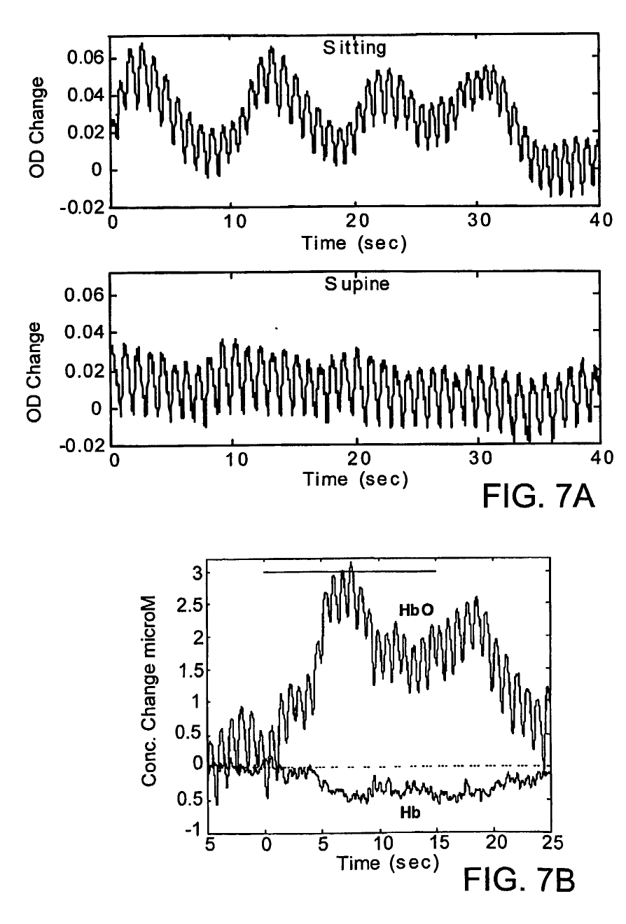

Baseline Recordings. FIG. 7A shows the observed amplitude modulations from a single subject during two different baseline conditions (in optical density units). The top trace—with data gathered at 830 nm from approximately location C3 in the international 10-20 system—shows 40 sec of eyes-closed baseline while the subject was seated upright. The bottom trace is from the same subject, again eyes closed baseline, but while lying supine. Heart pulsations are clear in both records, while in the upright case an additional high-amplitude oscillation of 0.1 Hz appears. The frequency of this periodicity corresponds to the Mayer wave—a systemic blood pressure oscillation that is more prominent when standing or sitting than when lying down (Taylor JA et al. Am J. Physiol. 1998 274:H1194-201.).

Motor Task. The data gathered from a single detector (again sampling from approximately location C3) for the motor task appear in FIG. 7B for a supine subject. The horizontal bar indicates the period of motor activity, while the upper and lower traces show the change in HbO₂ and Hb (respectively) from the rest period. These traces involve no averaging, only a boxcar smoothing function with a 2 Hz bandwidth. The expected hemodynamic response for this task, based on previous fMRI experiments, would be an increase in HbO₂ (and a concomitant decrease in Hb) starting ˜3 sec after motor activity onset, peaking around 6-9 sec post-onset, and decaying to baseline some 7-11 sec following the cessation of motor activity (Bandettini P A et al. Magn Reson Med. 1992 25:390-7.). Both optical time courses match this profile.

### Use of DOT to Record Cortical Activation

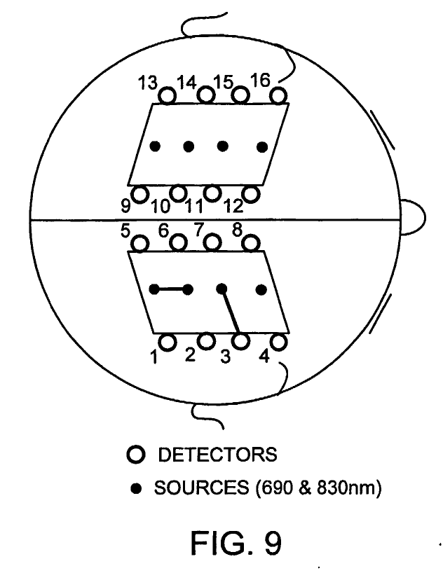

Experimental Setup. In one subject, we performed a pain stimulation task. We stimulated ˜1 cm² of the back of the subject's hand with a plate attached to a thermal stimulator. The temperature of the plate was set to 32° C. during baseline. During stimulation, it was raised to 47° C. in 2-3 sec. We alternated rest and stimulation four times during each run. We performed two runs stimulating the right hand and two runs stimulating the left hand. The optical probe was positioned on the subject's head to cover the left and right motor cortex. The probe geometry is shown in FIG. 9.

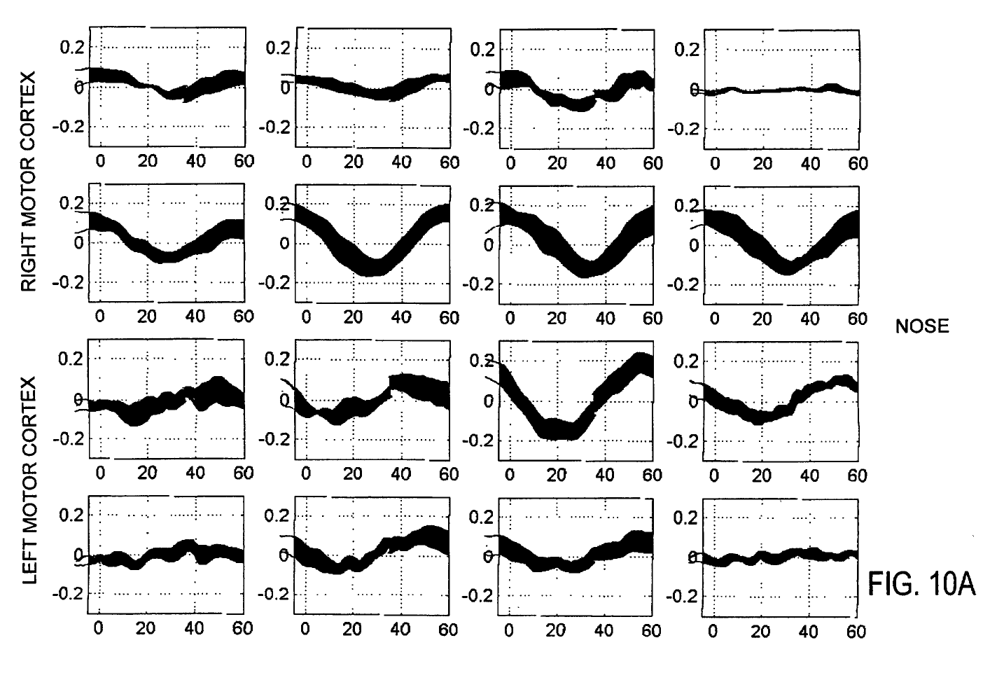

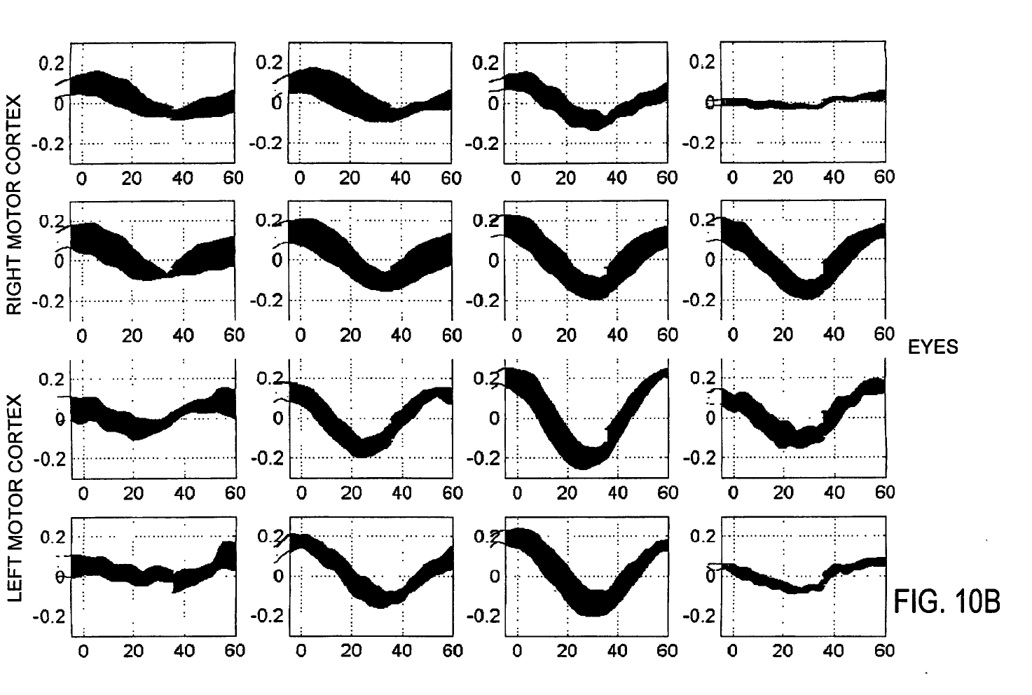

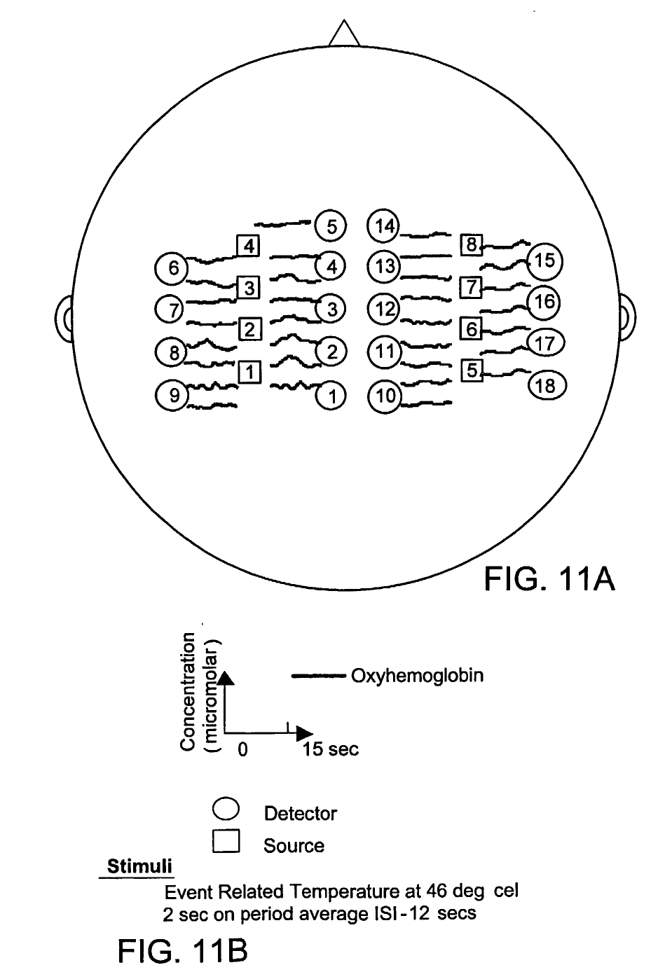

FIG. 10A shows the results for deoxy-hemoglobin during left hand stimulation and FIG. 10B shows the results for right hand stimulation. These results were obtained by filtering the optical data with a band pass filter between 0.016-0.3 Hz and averaging 6 stimuli. The thickness of the lines represents the standard error. The localized decrease in deoxy-hemoglobin is larger both in size and amplitude in the contralateral motor cortex. These data indicate that the method may be used to monitor pain in a subject. FIG. 11 shows results from another experiment.

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the appended claims.

Other embodiments are within in the claims.

What is claimed is:

1. A method for measuring a central nervous system (CNS) activity in a subject, the method comprising:
identifying a subject as a subject who experiences pain or analgesia; and
determining a level of activation in a cortical region of the subject by detecting a hemodynamic parameter in the subject, wherein the level of activation is indicative of the CNS activity in the subject.

2. The method of claim 1, wherein the pain is acute pain or chronic pain.

3. The method of claim 2, wherein the chronic pain is chronic nociceptive pain or neuropathic pain.

4. The method of claim 1, wherein the subject is anesthetized.

5. The method of claim 1, wherein detecting a hemodynamic parameter comprises performing diffuse optical tomography (DOT) and producing an image of the cortical region from which the level of activation is determined.

6. The method of claim 1, wherein the level of activation is determined relative to a reference level of activation.

7. The method of claim 6, wherein the reference level of activation is based on an assessment of the subject at different time.

8. The method of claim 1, further comprising administering an anesthetic or pain reductant if the level of activation is indicative of pain.

9. The method of claim 1, further comprising reducing the concentration an anesthetic in a subject if the level of activation is indicative of analgesia.

10. The method of claim 1, further comprising administering an anesthetic if the level of activation is indicative of pain.

11. The method of claim 1, wherein the SI cortical region is evaluated.

12. A method for identifying a therapeutic intervention for a neurological condition, the method comprising:
(a) administering a test intervention to a mammalian subject, wherein the subject has a neurological condition or is subjected to a stimulus capable of inducing CNS activity indicative of the neurological condition;
(b) performing diffuse optical tomography (DOT) on the subject; and
(c) determining a level of activation of a cortical region, wherein the level of activation is indicative of the effectiveness of the test intervention as a therapeutic intervention.

13. The method of claim 12, wherein the neurological condition comprises a psychiatric disorder.

14. The method of claim 13, wherein the psychiatric disorder comprises depression or anxiety.

15. The method of claim 12, wherein the neurological condition comprises pain.

16. The method of claim 12, wherein the stimulus is a noxious stimulus.

17. The method of claim 16, wherein the noxious stimulus is an acute painful stimulus.

18. The method of claim 12, wherein the stimulus is a non-noxious stimulus.

19. The method of claim 12, wherein the level of activation is determined relative to a control.

20. The method of claim 12, wherein the level of activation is determined relative to a control image of the cortical region of a control subject, wherein the control image is obtained during the presence of the stimulus, and the test intervention is not administered to the control subject, and wherein the control image is obtained using DOT.

21. The method of claim 12, wherein the test intervention comprises administering a test compound.

22. The method of claim 12, wherein the test intervention comprises administering a physical stimulus, an electrical stimulus, a thermal stimulus, electromagnetic radiation, or a surgical, medical, or dental procedure.

23. The method of claim 12, wherein the subject is experiencing chronic pain.

24. A method of evaluating a neurological condition in a subject, the method comprising:
(a) determining a level of activation in a cortical region of a mammalian subject by performing diffuse optical tomography (DOT) on the subject, wherein the level of activation is indicative of the neurological condition; and
(b) administering a therapeutic intervention to the subject to maintain or change the level of activation, thereby managing treatment of the neurological condition in the subject.

25. The method of claim 24, wherein the administering in step (b) maintains the level of activation.

26. The method of claim 24, wherein the administering in step (b) changes the level of activation.

27. The method of claim 24, wherein the DOT produces an image of the cortical region from which the level of activation is determined.

28. The method of claim 24, wherein the level of activation is determined relative to a control.

29. The method of claim 24, wherein the mammal is a human.

30. The method of claim 24, wherein the neurological condition comprises a psychiatric disorder.

31. The method of claim 24, wherein the neurological condition comprises pain or anesthesia.

32. The method of claim 31, wherein the pain is acute pain or chronic pain.

33. The method of claim 32, wherein the chronic pain is chronic nociceptive pain or neuropathic pain.

34. The method of claim 24, wherein the level of activation is determined relative to a control image of the cortical region of a control subject, wherein the control subject is not experiencing the neurological condition, and wherein the control image is obtained using DOT.

35. The method of claim 24, further comprising, prior to the determining step (a), administering a contrast agent to the subject, wherein the contrast agent is detected by DOT in step (a).

36. The method of claim 24, wherein the SI cortical region is evaluated.

37. The method of claim 24, wherein the subject is undergoing a surgical procedure.

38. The method of claim 24, wherein the neurological condition comprises anesthesia and the administering maintains anesthesia of the subject.

39. The method of claim 24, wherein the administering treats chronic pain.

40. The method of claim 24, wherein the therapeutic intervention comprises administering a pain reductant or anesthetic.

41. The method of claim 24, wherein the therapeutic intervention comprises administering a physical stimulus, an electrical stimulus, a thermal stimulus, electromagnetic radiation, or a surgical, medical, or dental procedure.

42. A method for correlating subcortical activity with cortical activity in a subject having a neurological condition, the method comprising the steps of:
(a) performing magnetoencephalography (MEG), single proton emission tomography (SPECT), positron emission tomography (PET), or functional magnetic resonance imaging (fMRI) on a first subject experiencing a neurological condition to determine a level of activation of a subcortical region;
(b) performing diffuse optical tomography (DOT) on a second subject experiencing the neurological condition to determine a level of activation of a cortical region; and
(c) comparing the level of activation of the subcortical region with the level of activation of the cortical region, thereby correlating subcortical activity with cortical activity.

43. The method of claim 42, wherein the subcortical activity is indicative of anesthesia or a psychiatric disorder.

44. The method of claim 42, wherein the subcortical activity is indicative of pain.

45. The method of claim 42, wherein the first and second subjects are the same.

46. The method of claim 42, wherein, in step (a), fMRI is performed.

47. A method for evaluating pain or analgesia in a subject, the method comprising:
evaluating activity in the primary somatosensory (SI) cortex of a subject by detecting a hemodynamic parameter, wherein activity in SI is indicative of pain sensation.

48. The method of claim 47 wherein the hemodynamic parameter is a parameter assessing blood oxygenation or blood flow.

49. The method of claim 47 wherein the subject is exposed to an external source of pain within 10 minutes of the evaluating.

50. The method of claim 47 wherein the subject experiences chronic pain or analgesia.

51. The method of claim 47 wherein the method further includes determining a baseline level of activity.

52. The method of claim 47 wherein changes in the hemodynamic parameter are detected using a light source and detector.

53. The method of claim 47 wherein changes in the hemodynamic parameter are detected using a plurality of detectors.

54. The method of claim 47 wherein changes in the hemodynamic parameter are detected using a plurality of sources and a plurality of detectors.

55. The method of claim 47 wherein information from the plurality of detectors is used to localize the site of neural activity to a region within SI.

56. The method of claim 47 wherein information from the plurality of detectors is used to create a projection of neural activity in a map of the cortex.

57. The method of claim 47 wherein the activity is compared to reference activity.

58. The method of claim 47 wherein the activity used to determine a baseline for the subject, and the subject's baseline is compared to a reference baseline.

59. The method of claim 47 wherein the reference activity is activity in a reference subject who is an individual not subjected to pain.

60. The method of claim 47 wherein the reference activity is activity in a reference subject who is an individual who suffers from chronic pain.

61. The method of claim 47 wherein the reference activity is activity in a reference subject who is an individual who has analgesia.

62. The method of claim 47 wherein the reference activity is activity in the subject under another condition.

63. The method of claim 47 wherein signal processing routines are used to process signals representative of the detected activity.

64. The method of claim 47 wherein the parameter is corrected for a systemic physiological response.

65. A method of providing a treatment to a subject, the method comprising:
providing a treatment to the patient;
evaluating the SI region of the patient for neural activity; and
altering the treatment as a function of activity in the SI region that indicates pain.

66. The method of claim 65, wherein the treatment comprises administering a pain reductant.

67. The method of claim 65, wherein the treatment comprises administering an anti-inflammatory agent.

68. The method of claim 65, wherein the treatment comprises acupuncture.

69. A method of providing anesthesia to a patient, the method comprising:
providing anesthesia to the patient;
evaluating the SI region of the patient for activity; and
altering the amount of anesthesia, if activity in the SI region indicative of pain is detected.

70. A method of preparing a subject for neurosurgery, the method comprising:
having the subject perform a motor task or providing a sensory input to the subject; and
evaluating hemodynamics in the cortex of the subject, thereby mapping a region of the cortex relative to the motor task or sensory input.

71. A computer-readable database comprising a plurality of records, each record comprising:
(a) one or more of the following: (i) information representing a subjective measure of pain or analgesia in a subject; and (ii) information indicating a pain relief treatment provided to the subject; and
(b) information representing SI activity in the subject.

72. A system for objectively evaluating pain or analgesia, the system comprising:
a light source and a detector, wherein the light source and the detector are configured for placement on the scalp of a subject, such that the detector can evaluate hemodynamic parameters associated with the cortex; and
a processor configured to evaluate signals from the detector for changes in deoxyhemoglobin as an indicator of neural activity in the cortex, and correlate signals with reference information to provide an objective measure of pain or analgesia based on activity in the cortex of a subject.

73. The system of claim 72 that comprises a plurality of light sources and a plurality of detectors.

74. The system of claim 72 wherein a plurality of light sources are arranged in a first row on one hemisphere of the scalp and a second row on the other hemisphere of the scalp.

75. The system of claim 74 wherein a plurality of detectors are arranged in four rows, one on either side of each row of light sources.

76. The system of claim 72 wherein the processor is further configured to produce an image map based on information from the detectors.
